(19) United States
(12) Patent Application Publication
(10) Pub. No.: US 2022/0387710 A1
(43) Pub. Date: Dec. 8, 2022

(54) DRUG DELIVERY DEVICE AND SYSTEM

(71) Applicant: AMGEN INC., Thousand Oaks, CA (US)

(72) Inventors: Shawn Malloy, Cambridge, MA (US); Nicholas J. Clark, Thousand Oaks, CA (US); Hillary Doucette, Cambridge, MA (US); Zaheer Darrell Abdool, Cambridge, MA (US)

(21) Appl. No.: 17/770,341

(22) PCT Filed: Oct. 22, 2020
(86) PCT No.: PCT/US2020/056832

§ 371 Date: Apr. 20, 2022

Related U.S. Application Data
(60) Provisional application No. 62/925,599, filed on Oct. 24, 2019.
(60) Provisional application No. 62/925,610, filed on Oct. 24, 2019.
(60) Provisional application No. 62/925,699, filed on Oct. 24, 2019.
(60) Provisional application No. 62/925,591, filed on Oct. 24, 2019.
(60) Provisional application No. 62/925,622, filed on Oct. 24, 2019.

(51) Int. Cl. A61M 5/168; A61M 5/172

(52) U.S. Cl. CPC A61M 5/16877; A61M 5/1723; A61M 2205/18; A61M 2205/6063; A61M 2205/3368; A61M 2205/3303

(57) ABSTRACT

A drug delivery system for delivering a drug product is provided that includes a drug product container containing a drug product, a fluid path adapted to receive the drug product from the drug product container, and a drug delivery device positioned along and/or adjacent to the fluid path. The drug delivery device may include a housing, a fluid displacement assembly at least partially supported by and/or surrounded by the housing, and a drive component at least partially supported by and/or surrounded by the housing. The drive component may drive the medicament through the fluid displacement assembly. Further, the drug delivery device includes a controller workingly coupled with the drive component for controlling the drug delivery device. The controller may operate in a normal operation mode and a reserve mode.

### CROSS-REFERENCE TO RELATED APPLICATIONS

The present application claims priority to U.S. Application Ser. No. 62/925,610, entitled “Drug Delivery Device and System,” filed Oct. 24, 2019; U.S. Application No. 62/925,591, entitled “Drug Delivery Device and System,” filed Oct. 24, 2019; U.S. Application No. 62/925,599, entitled “Device and Method for Temperature-Controlled Storage and Preparation of Drug Product,” filed Oct. 24, 2019; U.S. Application No. 62/925,622, entitled “Drug Delivery Device and System,” filed Oct. 24, 2019; and U.S. Application No. 62/925,699, entitled “Optimized Continuous IV Infusion IV-Bag and/or IV-Cartridge Design,” filed Oct. 24, 2019. The priority applications are hereby incorporated by reference in their entireties.

### FIELD OF DISCLOSURE

The present disclosure generally relates to drug delivery devices and, more particularly, to devices and/or systems for long-term, continuous, semi-continuous, and/or intravenous drug delivery.

### BACKGROUND

Drugs are administered to treat a variety of conditions and diseases. Intravenous (“IV”) therapy is a drug dosing process that delivers drugs directly into a patient's vein using an infusion contained in a delivery container such as IV bag, a glass vial, and/or other pliable bag, and tubing connected to a needle subsystem that fluidically communicates with the reservoir through the pump assembly collectively called infusion set. These drug dosings may be performed in a healthcare facility, or in some instances, at remote locations such as a patient's home. In certain applications, a drug delivery process may last for an extended period of time (e.g., for one hour or longer) or may include continuous or semi-continuous delivery of a drug over an extended period of time (e.g., for several hours, days, weeks, or longer). For many of these relatively long-term delivery requirements, a pump is often utilized to control and/or administer the drug to the patient. The pump may be coupled (physically, fluidly, and/or otherwise) to various components, such as a drug delivery container, supply lines, connection ports, and/or the patient. In some examples, a drug product may be shipped to a healthcare facility (e.g., an inpatient facility, an outpatient facility, and/or a pharmacy) in a powdered or lyophilized form.

It may be desirable to utilize a pump and/or overall system that is portable and/or wearable. It may also be desirable to utilize a pump and an overall system that minimizes patient inconvenience, minimizes the size and profile of the device and the overall system, minimizes the complexity of the device and overall system, minimizes the noise and vibration of the device, accommodates easy connection/disconnection and changeover of the infusion set, simplifies or automates priming of the line, accommodate easy delivery interruption and reestablishment based on required therapy and delivery profile, easily provides status of delivery and other important user information such as occlusion and volume of drug delivered or remaining in the reservoir, reduces the cost of the device and the overall system, increases the reliability and accuracy of the device and the overall system.

When reconstituting these drugs for administration, it is of particular importance to maintain a sterile environment so as to not taint or otherwise damage the quality of the drug. Additionally, some classes of drugs such as bi-specific T-cell engagers may require exceptionally accurate quantities of the drug product and/or other fluids required for dosing so as to prevent the drug product from becoming toxic. Oftentimes, the healthcare professional must prepare the drug by closely following a set of steps to ensure a sterile environment is maintained and that correct quantities of ingredients are added to the delivery container. When reconstituting these drugs for administration, it may be desirable or necessary to utilize a diluent, such as by adding a diluent to a drug product vial. As a result of these various steps and requirements, the reconstitution process may be time-consuming, tedious, and may have an unacceptable or undesirable error rate.

The current process of reconstituting a lyophilized oncology product is often done either at the hospital or the specialty compounding pharmacy by a licensed pharmacist. The use of a hood is often required to perform reconstitution steps to provide a sterile working environmental which can be cumbersome for pharmacist given the complexity of the steps. In addition, this reconstitution process involves the use of multiple needles to withdraw/add sterile water for injection (WFI), saline and/or Intravenous Solution Stabilizer (IVSS) solutions. Typically, for relatively complex oncology products such as a Bi-specific T-cell Engager (BiTE®) molecule (e.g. Blincyto®) prepared in an IV bag, a specified volume of WFI is added to reconstitute a lyophilized drug product contained in a vial via the use of a needle and syringe system. Then, the applicable volume of saline and IVSS solutions are added to an empty IV bag before the final reconstituted drug product is introduced. The overall process may involve relatively extensive manual labor time and steps. Often these steps include handling and/or use of needles, which may include inherent potential risks of needle-stick injuries.

As another potential step in preparing / storing a drug product for use, it may be desirable or beneficial to maintain a drug product at a particular temperature, such as a relative cold temperature for storage and/or a warmer temperature (e.g., room temperature) for drug administration.

As described in more detail below, the present disclosure sets forth components, systems, and methods for drug delivery systems and components for preparing and/or storing drug product that may embodying advantageous alternatives to existing systems and methods, and that may address one or more of the challenges or needs mentioned herein, as well as provide other benefits and advantages.

### SUMMARY

In accordance with a first aspect, a drug delivery system for delivering a drug product is provided that includes a drug product container containing a drug product, a fluid path configured to receive the drug product from the drug product container, and a drug delivery device positioned along and/or adjacent to the fluid path. The drug delivery device may include a housing, a fluid displacement assembly at least partially supported by and/or surrounded by the housing, and a drive component at least partially supported by and/or surrounded by the housing. The drive component may drive the medicament through the fluid displacement assembly. Further, the drug delivery device includes a controller workingly coupled with the drive component for controlling the drug delivery device. The controller may operate in a normal operation mode and a reserve mode.

In accordance with a second aspect, a drug delivery system for delivering a drug product is provided. The drug delivery system includes a drug product container containing a drug product, a fluid path adapted to receive the drug product from the drug product container, and a drug delivery device positioned along and/or adjacent to the fluid path. The drug delivery device includes a housing, a fluid displacement assembly at least partially supported by and/or surrounded by the housing, a drive component at least partially supported by and/or surrounded by the housing, and a controller workingly coupled with the drive component to control the drug delivery device. The drive component is adapted to drive the medicament through the fluid displacement assembly. The controller is adapted to send a user output when a low drug event has occurred.

In accordance with a third aspect, a drug delivery system for delivering a drug product includes a drug product container containing a drug product, a fluid path to receive the drug product from the drug product container, a drug delivery device positioned along and/or adjacent to the fluid path, and a scannable identifier tag coupled with at least the drug product container, the fluid path, and/or the drug delivery device. The scannable identifier tag provides information regarding the drug product container, the fluid path, and/or the drug delivery device.

In accordance with a fourth aspect, a device for storing a drug product container in a temperature-controlled state includes a container positionable in at least an open and closed configuration, a temperature element adjustable between a heating mode and a cooling mode to selectively heat and/or cool the container, and an ultraviolet element to reduce a bioburden level of at least a portion of the container.

In accordance with a fifth aspect, a drug delivery system for delivering a medicament to a user includes a medicament container a fluid path adapted to at least selectively fluidly connected the drug product container and the user, a sensor positioned adjacent and/or on the user and adapted to measure cytokine levels, and a drug delivery device positioned adjacent to and/or along the fluid path. The drug delivery device includes a housing, a pump coupled with the housing, a drive component to drive the medicament through the pump, and a controller workingly coupled with the drive component. The controller adjusts at least one parameter of the drive component based on input information received from the sensor.

In accordance with a sixth aspect, a flexible drug product container for a drug delivery system having an intravenous tube includes a body portion adapted to contain a drug product, a spike-free IV-set attachment port, and a sterile-disconnect and custom pump head integration component.

### BRIEF DESCRIPTION OF THE DRAWINGS

The above needs are at least partially met through provision of the systems and approaches for drug delivery and drug delivery device reconstitution described in the following detailed description, particularly when studied in conjunction with the drawings, wherein:

FIG. 1 illustrates an example drug delivery device in accordance with various embodiments;

FIG. 2 illustrates a partial cross-section of an example drug delivery device in accordance with various embodiments;

FIG. 3 illustrates an exploded view of an example drug delivery device in accordance with various embodiments;

FIG. 4 illustrates an exploded view of an example drive assembly for a drug delivery device in accordance with various embodiments;

FIG. 5 illustrates an exploded view of an example pump head for a drug delivery device in accordance with various embodiments;

FIG. 6 illustrates an exploded view of an example pressure sensor assembly and manifold assembly for a drug delivery device in accordance with various embodiments;

FIG. 7 illustrates an exploded view of an example PCA and battery assembly for a drug delivery device in accordance with various embodiments;

FIG. 8 is a flow chart for an example controller for a drug delivery device in accordance with various embodiments;

FIG. 9 illustrates an example drug delivery system in accordance with various embodiments;

FIG. 10 illustrates an example drug delivery device in accordance with various embodiments;

FIG. 11 illustrates an example drug delivery system having an integrated reader/writer in accordance with various embodiments;

FIG. 12 illustrates an example temperature-controlled drug storage device in a first position in accordance with various embodiments;

FIG. 13 illustrates the example temperature-controlled drug storage device of FIG. 12 in a second position in accordance with various embodiments;

FIG. 14 illustrates an alternative example drug delivery system having a first example sensor for monitoring a user in accordance with various embodiments;

FIG. 15 illustrates an alternative example drug delivery system having a second first example sensor for monitoring a user in accordance with various embodiments;

FIG. 16a illustrates an alternative drug product container in accordance with various embodiments;

FIG. 16b illustrates an alternative drug product cartridge in accordance with various embodiments;

FIG. 16c illustrates an alternative drug product container and cartridge in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The present disclosure relates to a drug delivery devices and systems and, more particularly, to systems having a pump and a system for long-term, continuous, semi-continuous, and/or intravenous drug delivery. Under some conditions, a drug delivery process may last for an extended period of time (e.g., for one hour or longer) or may include continuous or semi-continuous delivery of a drug over an extended period of time (e.g., for several hours, days, weeks, or longer) or may include delivery via an intravenous connection to a patient. The present disclosure utilizes various features for potentially improved flexibility and user convenience in changing out drug product containers for extended, continuous, semi-continuous, and/or intravenous delivery, in addition to improved drug dose accuracy and/or improved pump controls, while maintaining a relatively compact sized system that may be desirable or appropriate

In some examples, the drug delivery device may include a controller capable of operating in a number of modes. More specifically, the controller may operate in a normal operation mode until the drug product reaches a certain, predetermined level and/or until the device has been in operation for a certain, predetermined amount of time, in which case the controller may then switch to reserve mode of operation. During the reserve mode, the device may output user notifications, such as haptic feedback (e.g., vibration), visual alerts (e.g. flashing screen or messages), and/or audio feedback (e.g., alarm chimes or other audio feedback). The user notifications may occur with increasing frequency and/or volume/intensity until a user changes the drug product container and/or clears the alarm. During reserve mode, the device may also decrease (e.g., “ramp down”) the drug dosage to extend the amount of time before the drug product container is empty.

With some drug delivery configurations and/or drug products, it may be important and/or desirable to deliver drug product in a continuous or near-continuous manner, without interruption. This may particularly be the case with canonical BiTE®-based therapies. A potential critical juncture that may interrupt the therapy may occur when the drug reservoir is exhausted and requires changing with a fresh cassette or container. To ensure the user correctly accounts for and handles the situation, a set of software tools may be implemented into the system. Additionally, or alternatively, in some examples, the drug reservoir may be overfilled to account for potential lapses in judgment to change the reservoir (e.g., a labeled volume of 200 mL may have an extra 20 mL or 10% overfill of drug). The controller and/or software implemented thereon may indicate a run down to zero when the labeled quantity of drug product has been dispensed. The controller may then trigger escalating alarms to ensure the user knows this to be the case. Should the user be unable to replace the drug reservoir by the time the labeled volume is exhausted, the pump may automatically enter “Emergency Reserve Mode” with the 20 mL of overfill available. In this mode, the user may either have a predetermined amount of time to change the reservoir, or may input how much time they need before they can change the reservoir. Based on the amount of time inputted, the controller may cause the pump to ramp down the rate of infusion to ensure the reserve amount of drug is not exhausted until said time the user has indicated they can change the reservoir with a fresh one. User feedback throughout the mode may increase the likelihood that the user is continuously reminded that the reservoir needs to be changed.

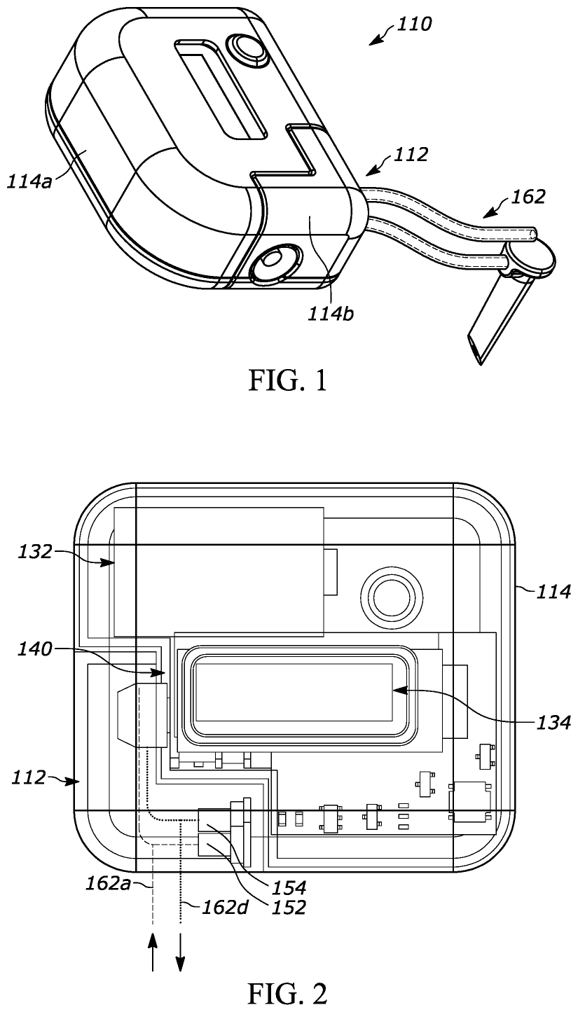

Turning to the figures, FIGS. 1 and 2 show a drug delivery device such as a pump 110 having, generally, a pump head 112 having a durable or reusable housing 114a, disposable housing 114b, a fluid flowpath 162, a power source such as a battery 132, a drive assembly such as a motor 140, a controller and display 134, and a pair of pressure sensors (e.g., inlet pressure transducer 152 and outlet pressure transducer 154). The two housing components 114a, 114b cooperate to define the overall housing 114. In some examples, the durable or reusable housing 114a may be disposable as suitable. Similarly, in some examples, the disposable housing 114b may be reusable, although certain sterilization and/or refurbishment steps may be required or desirable to achieve this reusability.

As is further illustrated in FIG. 2, a medicament from a drug product container may travel through an input tube, into the pump head 112, and out of the pump through an output tube. In other words, the pump is able to urge the medicament through the pump head 112. While the pump head 112 shown in FIG. 2 is a peristaltic pump but other suitable configurations may be used, such as a positive displacement pump. The pump head 112 shown in FIGS. 1 and 2 is a ring pump that utilizes a generally circular-shaped loop of tubing 162 to create peristaltic forces. As a more specific example, the pump head 112 has a component that pinches or otherwise occludes the ring-shaped tube section in a circular motion to urge fluid through the tube 162.

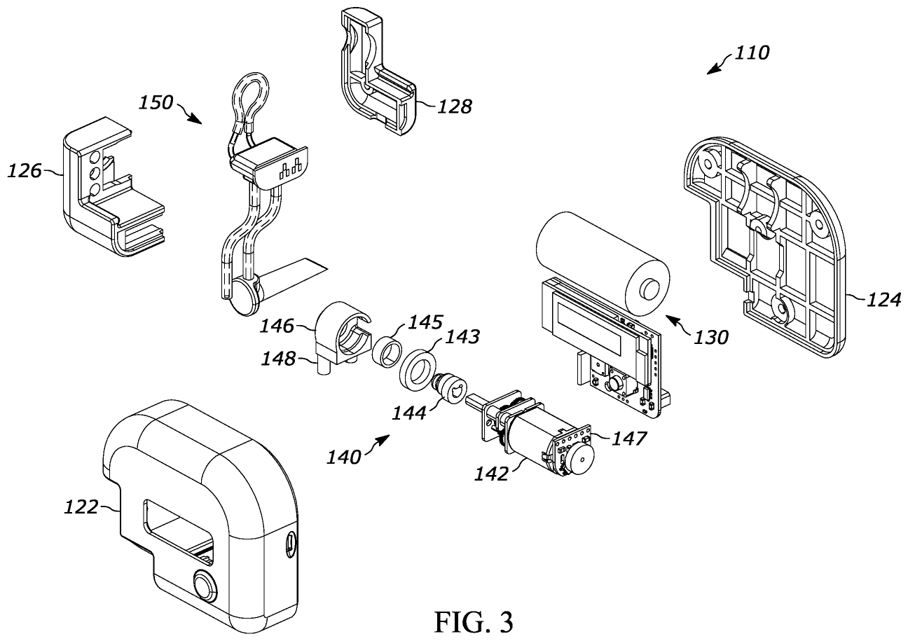

FIG. 3 shows an exploded view of the pump 110, including sub components of the housing 114, such as a controller front case 122, a controller rear case 124, a pump head front case 126, and a pump head rear case 128. These four components 122, 124, 126, 128 generally fit together to form at least the majority of the housing 114. These four components 122, 124, 126, 128 may be made of a generally rigid and lightweight material, such as plastic, a composite, or any other suitable material. The front/rear paired components (122, 124 on one hand, and 126, 128 on the other) may fit together via fasteners, snap-fit connections, an adhesive, or any other suitable coupling components/methods. A PCA and battery assembly 130 is at least partially contained within the housing 114, with a display screen 134 (FIGS. 2 & 7) defining a portion of the housing 114.

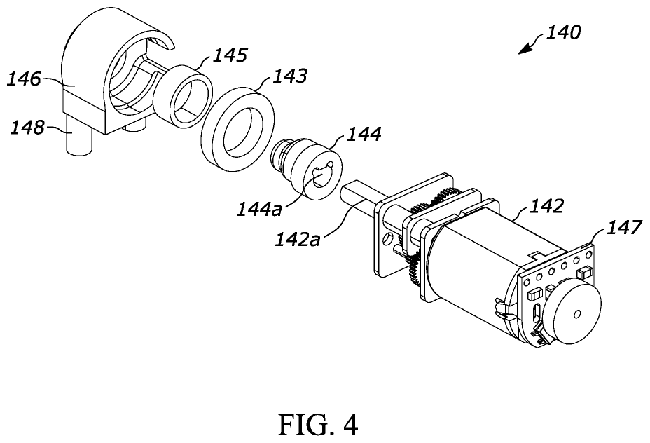

FIG. 3 further shows an exploded view of the drive assembly 140 (e.g., the motor assembly), a tube set, and pressure sensors 150. With reference to FIGS. 3 and 4, the drive assembly 140 generally includes a motor 142, a retainer ring 143, an eccentric hub 144, a sleeve bearing 145, a pump race 146, an encoder board 147, and a generally pliant/flexible isolation mount or mounts 148. The motor 142 provides a rotational driving force. The retainer ring 143 retains other components in the housing (namely the tubes, as discussed more below) and/or for aligning the eccentric hub 144. The eccentric hub 144 utilizes a cam feature to generate peristalsis. The sleeve bearing 145 provides a barrier between the eccentric hub 144 and the tubing (such as the ring tube 158). The pump race 146 is adapted to house the previously-described circular shaped tube section. The encoder board 147 is configured to measure an actual speed of the motor for increased accuracy and precision. The generally pliant/flexible isolation mounts 148 prevent part misalignment, reduce drive torque/power, and provide compliance for head installation.

As illustrated in FIGS. 3 and 4, the isolation mounts 148 allow compliance to the pump head 112. The isolation mounts 148 may be made of rubber or any other suitable material. The eccentric hub 144 includes a key portion 144a that receives a correspondingly shaped drive shaft 142a. Additionally, as shown in FIG. 3, the eccentric hub 144, the drive shaft 142a, the motor 142, and the encoder board 147 are located within the durable housing 114a of the pump 110, whereas the retainer ring 143, the sleeve bearing 145, and the pump race 146 are all located within the disposable housing 114b or the removable pump head 112. When the pump head 112 is coupled with the durable portion 114 of the pump 110, the eccentric hub 144 aligns with and is received within the retainer ring 143. During operation, as the drive shaft 142a of the motor rotates, the eccentric hub 144 rotates about an axis that is off-set from the drive shaft axis, thereby applying an annular, outward force onto the circular-shaped tube section positioned within the pump race 146. More specifically, the retainer ring 143 fits around the circumference of the eccentric hub 144. As the eccentric hub 144 rotates, it may cause the retainer ring 143 and/or the sleeve bearing 145 to press on a relatively discrete portion of the circular-shaped tube section, thereby compression and/or occluding that section of the tube. As the eccentric hub 144 (and the sleeve bearing) rotate further, the portion of the outer surface of the retainer ring 143 and/or the sleeve bearing 145 that is compressing the tube “rolls” around the inside of the pump race 146 and urges fluid in the tube to travel away from the pump head 112.

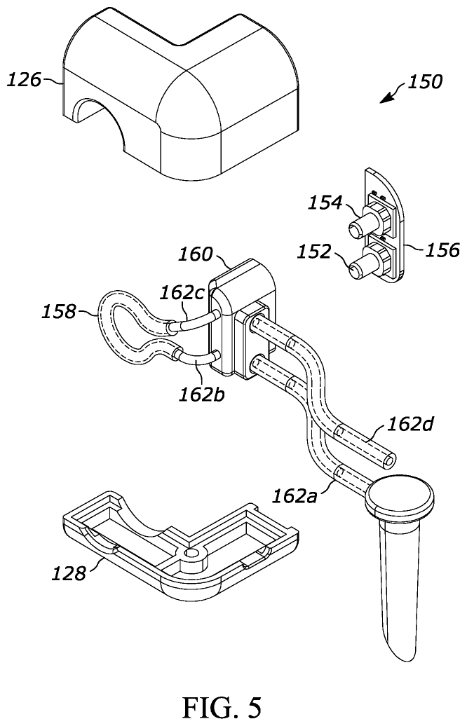

FIG. 5 shows the tube set and pressure sensors 150 in more detail, namely an exploded and enlarged view. FIG. 5 illustrates two sensors, namely inlet pressure transducer 152 and outlet pressure transducer 154, which measure fluid pressure in inlet and outlet portions of the flowpath 162. The respective transducers 152, 154 shown in the figures make contact with the flow in a tubing manifold 160 of the pump head 112. The tubing may be bonded to the tubing manifold 160. As a more specific example, the transducers 152, 154 are electrically connected to the pump controller via sprung connector contacts and directly measure the pressure in the flow at the inlet and outlet locations 162a, 162d. As an even more specific example, each transducer 152, 154 is electrically connected to a pressure transducer board 156 that is electrically connected to other electronic controls such as the motherboard (discussed below). For example, the transducers 152, 154 shown in the figures are each mounted on the pressure transducer board 156.

Each transducer 152, 154 shown in the figures may include a diaphragm, made from the same material as the tubing, placed inline on both the inlet and outlet tubes (162a, 162b). These diaphragms are located in the pump head 112 and make contact with a portion of the pump controller (e.g., the pressure transducer board) when the pump head assembly is installed via the pressure transducer board 156. At the point of diaphragm contact, load cells in the pump controller monitor variation in force exerted by the diaphragm which correlates to pressure changes in the flow. In this manner, the flow rate can be monitored at the inlet and outlet of the pump head 112 which provides the pressure sensor benefits discussed herein without introducing any new materials into drug contact. In some examples, one or both of the transducers 152, 154 may be workingly connected to the controller such that the controller is able to detect a disconnection of the fluid path based on changes detected by/values measured by the transducers. Other or alternative types of pressure sensors may be utilized, such as non-contact pressure sensors design to provide the benefits of pressure sensors but without the risk of material non-compatibility.

FIG. 5 also shows an example of the fluid flowpath 162 in more detail. For example, the fluid flowpath 162 may include an external tubing inlet side portion 162a, an internal tubing inlet side portion 162b, an internal tubing outlet side portion 162c, and an external tubing outlet side portion 162d. The various portions of tubing 162a-d may be integrally formed (i.e. a single piece of tubing), or they may be made of two or more sections of tubing that are fluidly connected with each other. The external tubing portions 162a, 162d shown in the figures may each be constructed from the same type and sized tubing as each other and potentially the same type and sized tubing as IV lines. In some examples, the internal tubing portions 162b, 162c may each be constructed from a smaller diameter tube to facilitate pressure measurement. The flowpath 162 also include a fluid displacement assembly, such as a ring tubing 158, i.e., the previously-discussed generally circular portion of tubing that is housed within the pump race 146. In one embodiment, the ring tubing 158 defines the boundary between the inlet fluid flowpath and the outlet fluid flowpath. As previously noted, the pump head 112 components depicted in FIG. 5 are supported by the pump head front and rear case 126, 128 and the pump head 112 is removably coupled with the remainder pump structure. The pump head 112 may be disposable and the remainder pump structure may be reusable (e.g. “durable”).

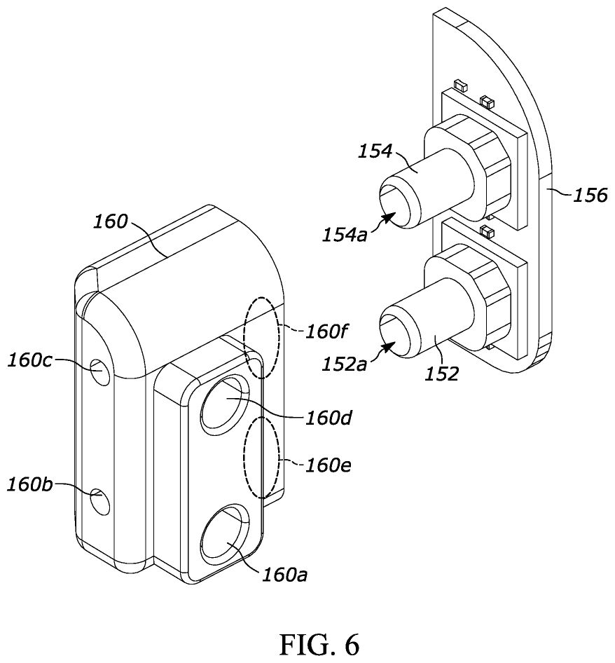

With reference to FIG. 6, an enlarged view of the tubing manifold 160 and the pressure sensors (152, 154, 156) is provided. The transducers 152, 154 are inserted within transducer ports (shown with dotted lines 160e and 160f) on a side of the manifold 160 so as to measure fluid pressure within the tubes that extend through the manifold 160. For example, the manifold 160 includes a manifold port external inlet 160a for receiving the external tubing inlet side portion 162a, a manifold port internal inlet 160b for receiving the internal tubing inlet side portion 162b, a manifold port internal outlet 160c for receiving the internal tubing outlet side portion 162c, and a manifold port external outlet 160d for receiving the external tubing outlet side portion 162d. The transducer ports 160e, 160f are in-line with the other ports and are sized to receive the transducers 152, 154 and may be in fluid communication with the other ports. For example, ports 160a, 160b, and 160e are in fluid communication with each other such that the inlet fluid flow travels through the tubing 162a, into contact with the diaphragm (the location of which is indicated by arrow 152a) of transducer 152, into the tubing 162b, through the ring tubing 158, through the tubing 162c, into contact with the diaphragm (the location of which is indicated by arrow 154a) of transducer 154, and into the tubing 162d.

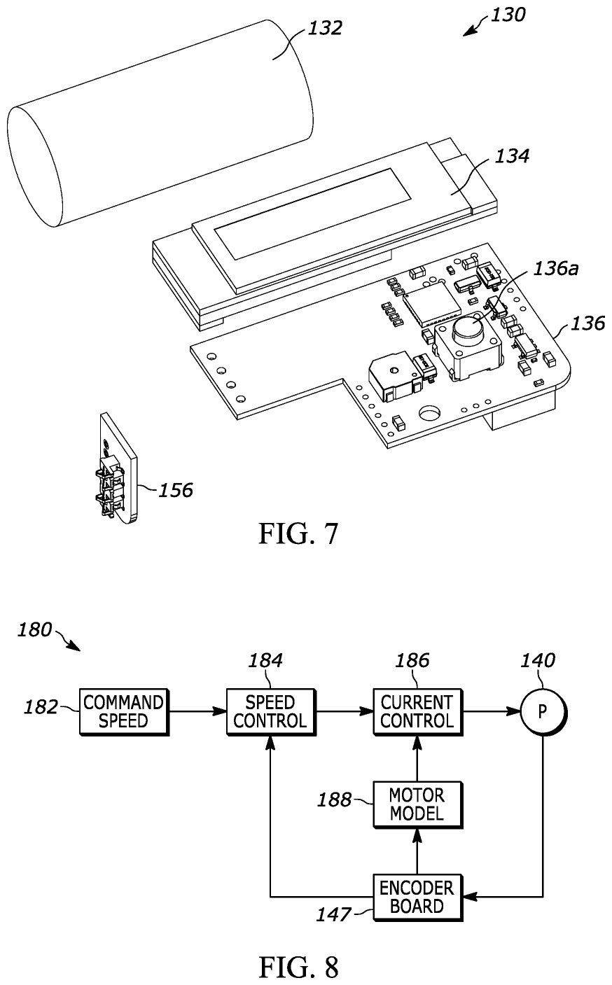

FIG. 7 illustrates the PCA and battery assembly 130, which in some examples includes a battery 132, an OLD display 134 (or alternatively, a LED, OLED, or LCD display), a motherboard 136, and the pressure transducer board 156. As discussed above, the pressure transducer board 156 is electrically coupled with the motherboard 136 such that the respective components may exchange inputs and outputs. The OLD display 134 may display user and/or pump information. The motherboard 136 may include an input in the form of a push button 136a electrically coupled therewith for operating the device, such as triggering a start or stop cycle. The motherboard 136 may host different functions and/or controls such as motor control; pulse with modulation (PWM) control; Proportional, Integral, Derivative (PID) control to stabilize the pump, sound control, user input control, and encoder board/speed control.

FIG. 8 illustrates a flowchart of an example operation of a controller 180 of the pump 110 that has improved accuracy and/or precision. The controller 180 may include the motherboard 136 and/or other components. For example, the controller 180 shown in FIG. 8 includes an encoder-fed, closed loop system. As a more specific example, the controller 180 includes an encoder board 147 for determining measured drive speed and a motor model 188 for determining a calculated drive speed. In this example, the controller 180 is configured to adjust at least one parameter of the drive component based on the measured drive speed and the calculated drive speed. For example, during operation, the motherboard 136 and/or a user input may dictate a desired speed for the motor 140, i.e., a “command speed” 182. The command speed 182 is then inputted into a “speed control” 184 component that may be electrically coupled with or integrally formed in the motherboard 136. The speed control 184 then sends input to the “current control” 186 which in turn sends a certain amount of current to the motor 140. An encoder board 147 is mounted to a portion of the motor 140 or adjacent to a portion of the motor 140 to measure the speed of a rotary portion of the motor. This measured speed information (e.g. measured drive speed) is then inputted back to the speed control 184. At the same time, the encoder board 147 inputs the measured speed information to a motor model 188, which calculates a calculated (or predicted) drive speed based on operating conditions. The speed control 184 and the current control 186 each are able to receive and process these inputs and potentially vary their operation based on the same. For example, if the calculated speed from the encoder board 147 differs from the command speed 182, then the respective components may be able to adjust the current level sent to the motor 140 to more accurately and precisely operate at (or near) the command speed. The controller 180 may also receive inputs from the pressure sensors 152, 154 as part of the feedback/control system.

This feedback/control system may allow the pump 110 to operate at a high accuracy. For example, the controller 180 may be configured such that the pump 110 is able to deliver medicament at an accuracy rate of at least 95%. More specifically, the controller 180 may be configured such that the pump 110 is able to deliver medicament at an accuracy rate of at least 97%. Even more specifically, the controller 180 may be configured such that the pump 110 is able to deliver medicament at an accuracy rate of at least 98%. Even more specifically, the controller 180 may be configured such that the pump 110 is able to deliver medicament at an accuracy rate of at least 99%. The controller 180 may be configured such that the pump 110 is able to deliver medicament at one or more of these accuracy levels during delivery of a dose of the medicament having a volume of at least 200 milliliters or 250 milliliters. This feedback/control system may allow the pump 110 to operate at a high efficiency, thereby maximizing battery life, reducing device noise and vibration, reducing generated motor heat, and/or improving overall performance. The feedback/control system may allow the pump 110 to operate at the accuracy levels discussed herein despite varying operating conditions, such as vertical height differential (positive or negative) between the pump 110 and the drug product container. For example, the pump 110 has been tested to maintain accuracy at a distance of +/−36 inches between the pump 110 and the drug product container.

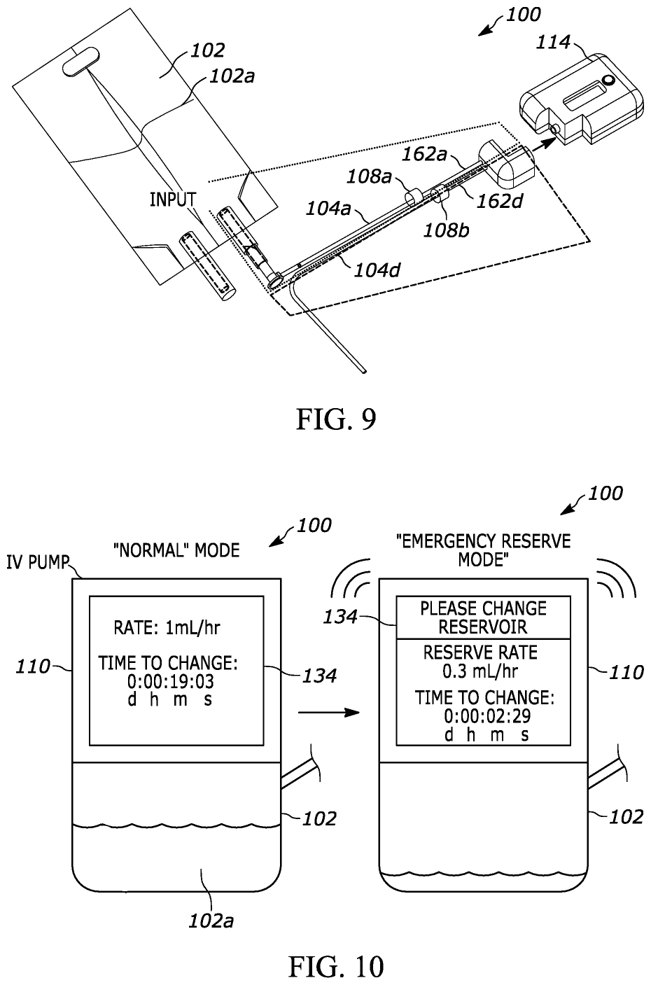

FIG. 9 shows an example drug delivery assembly 100 (or “system”) for use with the pump 110 that includes a number of connection points. For example, the assembly 100 shown in FIG. 9 includes a drug product container 102 which, in the illustrated example, is in the form of an IV bag for containing a drug product 102a (or medicament), an IV input line 104a, an IV output line 104d, a pair of adaptors 108a, 108b, and the tubing portions 162a, 162d leading to and from the pump 110. As a more specific example, the connection points may include quick-connect sterile connectors with respective sub-components that selectively mate with each other while maintaining sterility or another desirable cleanliness standard. For example, the quick-connect sterile connectors may snap or twist or screw together, they may have sheathed or covered components that become unsheathed or uncovered upon connection, and/or they may have Luer Lock or modified Luer Lock configurations. As another example, the connectors may include one or more stake connectors for coupling one of the tube portions 162 with the drug product container 102. The distal end of the IV output line 104d may also include or be coupled with a drug delivery connector (not shown) such as a needle, a Luer lock component, or another suitable component. As shown in FIG. 9, an IV spikes may pierce the port of the drug container 102 to physically connect the drug product container to the fluid path assembly 160.

In some examples, the adaptors may be sterile quick-connect components. Example CSTD devices may include the OnGuard CSTD provided by B. Braun Medical Inc, BD PhaSeal CSTD components, Equashield CSTD, Codon CSTD, and the like. Further, non-closed system transfer devices may be used such as West Pharmaceuticals vial and bag adapters. Other examples are possible. The prefilled delivery container may include any number of delivery container adapters having different specifications (e.g., port sizes) to accommodate the use of different drug product vials.

FIG. 10 shows an example pump 110 operating in “normal” mode and “emergency reserve” or “reserve” mode. The controller 180 may operate in the normal operation mode until the drug product 102a reaches a certain, predetermined level and/or until the pump 110 has been in operation for a certain, predetermined amount of time, in which case the controller 180 switches to reserve mode. The controller 180 may utilize different components or methods for determining the drug product level, such as component(s) for weighing the drug product container 102, volumetric flow sensors that measure the volume of drug product 102a that has flowed through the pump assembly 110, pressure sensors (that allow the controller to calculate the volume of drug product 102a that has flowed through the pump assembly 110), a timer that is directly programmed by a user or health care provider, or an automatic timer that is programmed by the controller 180 based on user inputs such as desired flowrate, volume of drug product 102a in the container 102, and desired “low volume” level.

During the reserve mode, the pump 110 may output user notifications, such as haptic feedback (e.g., vibration), visual alerts (e.g. flashing screen or messages on the display 134), and/or audio feedback (e.g., alarm chimes or other audio feedback). The user notifications may occur with increasing frequency and/or volume/intensity until a user changes the drug product container 102 and/or clears the alarm.

During reserve mode, the pump 110 may also decrease (e.g., “ramp down”) the drug dosage to extend the amount of time before the drug product container 102 is empty. For example, for some drug product therapies, it may be more desirable to continue drug delivery at a less-than-optimal flow rate than to discontinue drug delivery altogether, even for a short period of time. In such cases, a temporary ramp down dose may be more desirable than a normal dose rate followed by an extended period of time without any drug delivery (e.g., a time period longer than a typical bag change process). This may particularly be the case with canonical BiTE®-based therapies.

Additionally, or alternatively, the drug product container 102 can be overfilled to account for inadvertent lapses in judgment to change the drug product container 102 (e.g., a labeled volume of 200 mL may have an extra 20 mL or 10% overfill). The controller 180 may indicate a run down of the drug product 102a to zero when the labeled volume (e.g., 200 mL) has been dispensed. Escalating alarms would help ensure the user knows this to be the case. Should the user be unable to replace the drug product container 102 by the time the labeled volume is exhausted, the pump 110 may automatically enter “Emergency Reserve Mode” by using the 20 mL of available overfill drug product 102a. In this mode, the user would either be given a predetermined amount of time to change the reservoir, or the user would input how much time they need before they can change it. Based on the amount of time inputted, the pump may also ramp down the rate of infusion to ensure the reserve amount of drug product 102a is not exhausted until said time the user has indicated they can change the drug product container 102 with a replacement. User feedback throughout the mode may increase the likelihood that the user is continuously reminded that the drug product container 102 needs to be changed.

In some examples, the system 100 may be provided with a scannable identifier tag coupled with the drug product container to provide varying information such as, for example, a drug product date of manufacture, a drug product expiration date, a drug product volume, a drug product lot number, a drug product model number, a drug product region, and/or a drug product temperature information. More specifically, BiTE® therapy administration may be more complex than some other therapies and may require or benefit from understanding numerous variables to ensure the drug product 102a is safely and effectively administered. The device 110 is a central component in controlling aspects of drug administration. The therapies may involve many different parameter variables, and they may be subject to human errors and/or time consuming to manually input into the pump controller. Having a central scannable code (such as an RFID reader/writer) on the pump may facilitate automation and/or reduce errors through several functions.

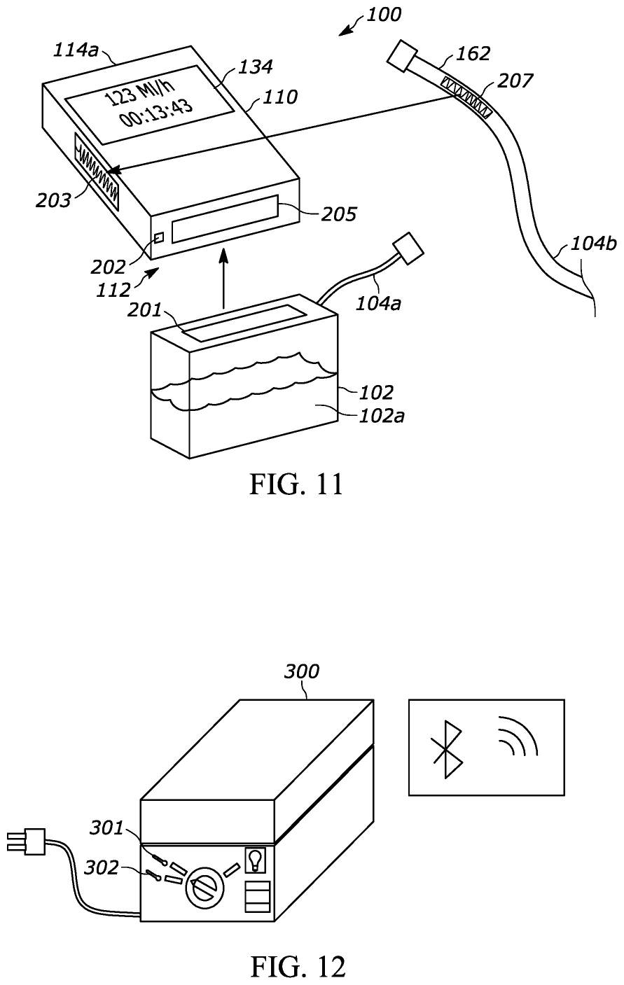

As illustrated in FIG. 11, any number of scannable identifier tags 201 may be coupled with at least the drug product container 102, the fluid path 162, and/or the drug delivery device 110. For example, FIG. 11 shows an identifier tag 201 positioned on the drug product container 102, an identifier tag 203 positioned on the durable housing 114a of the drug pump 110, an identifier tag 205 positioned on the removable pump head 112 of the drug pump 110, and an identifier tag 207 positioned on the fluid flow path 162 of the output line 104b.

The identifier tags 201, 203, 205, and 207 may include a scannable portion that permits a scanner and/or reader to obtain information from the tag. For example, the scannable portion may be an RFID chip, a QR code, a bar code, or any other suitable component for conveying information. The system 100 may be coupled with a reader/writer, such as via a computer, smart phone, or other suitable electronic component(s). Alternatively or additionally, the reader/writer may be integrated into the drug delivery device 110 so external software and/or hardware are not necessary. The scannable identifier tag 201, 203, 205, 207 may include information and/or functions such as, for example, recognize legitimate versus counterfeit drug product (i.e., authenticating information), recognize used versus new drug containers, track and create records of an amount of drug dispensed from the drug product container, recognize drug product expiration, calibrate prime volumes and flow rates based on tubing sets used, create therapy logs of drug usage over time, recognize and prevent reuse of old or used drug product containers, and/or recognize correct IV-line use. Other examples are possible.

Such a system may be implemented using a number of approaches. For example, as illustrated in FIG. 11, the drug delivery device 110 may be used as the RFID read/writer by incorporating a sensor 202 at the base or side where the drug product container 102 is connected therewith. Read/writable RFID tags may be incorporated into the manufacture of the drug reservoir with a number of values written to the drug product container 102 tag upon manufacture including, for example, a date of manufacture, a date of expiration, a fill volume, a lot number, a model/regional number, and/or a temperature probe (e.g., a temperature log to provide the patient and/or healthcare professional information as to how long the drug product container 102 has been at room temperature). Other examples are possible.

In some examples, the IV input and/or output lines 104a, 104b may be customized and produced specifically for use with a pump or pumps types with specific the material requirements (i.e., PVC free). In these examples, the device 110 may require scanning an RFID tag embedded in the IV line 104a, 104b that would prevent infusion until proper completion of the desired action to prevent use of incompatible lines. Additionally, the RFID tag on the IV line 104a, 104b may include information regarding manufacture thereof, such as, for example, a date of manufacture, a date of expiration, a lot number, a model/regional number, an inner diameter of the line, a length of the line, and/or the number of hours in use (to assist with alerting the patient and/or healthcare provide when it is appropriate to change the line 104a, 104b). Other examples are possible.

The information relating to IV tube inner diameter and length may be especially useful during calibration of the infusion prime volumes and flow rates of the device 110 specific to requirements based on the line being used. Additionally, the system 100 may facilitate automation and/or reduce errors through several functions.

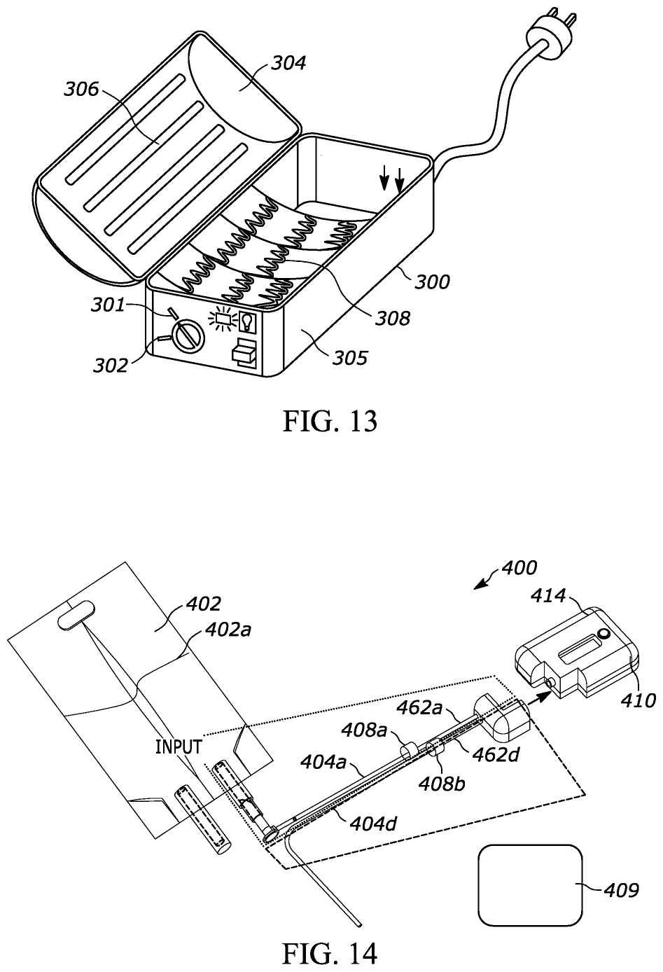

With reference to FIGS. 12 and 13, enabling patients to receive infusion therapy at home may be improve the patient experience for various therapies. In the case where a patient receives a pre-prepared, refrigerated IV-bag ahead of a planned therapy, an IV-bag warmer may enable the best in-home therapy experience by reducing the preparation time ahead of the infusion. Additionally, the device may contain a UV-light to briefly expose IV-bag/cartridge to UV light and potentially decrease microbial count(s).

For example, it may be desirable to store a drug product in cold storage conditions to preserve the drug product. It may also be desirable to warm a drug product to a warmer temperature, such as room temperature, before administering the same. As a more specific example, injecting cold therapies into a patient's body may lead to patient discomfort and/or other undesirable effects. As an even more specific example, it may be desirable to allow or cause a drug product to reach room temperature before injecting it into a patient's body.

As shown in the figures, a temperature-controlled device 300 has a closed configuration (FIG. 12) and an open configuration (FIG. 13). The temperature-controlled device 300 may be used in a heating configuration (indicated by a the heat symbol 301) and a cooling configuration (indicated by a cooling symbol 302). The heating configuration may be used as a bag warming option to a user patient to rapidly warm a previously-refrigerated drug product container and drug product to room temperatures. Additionally, the device 300 may have wireless connectivity to communicatively couple with a users or health care providers desired computing device. Additionally, in some examples, if the device 300 is in the heating configuration for a time period beyond a designated value, then the device 300 may be configured to switch to the cooling mode to preserve the drug product until the user can return the drug product container to a refrigerator and/or use the device 300 as a refrigerator.

As illustrated in FIG. 13, a top portion 304 of the device 300 (e.g., an inside surface of the top portion 304) may include a UV element 306. The UV element may reduce a bioburden level of the container and/or the drug product container disposed therein. Further, a bottom portion 305 of the device 300 (e.g., an inside surface of the bottom portion 305) may include a temperature element (e.g., the heating element/cooling element 308). The heating/cooling element 308 may be a single element that is able to heat and/or cool, or it may be two different elements that each have a dedicated function (heating or cooling).

As previously noted, the drug product container (not illustrated in FIGS. 12 & 13) may be in the form of an IV bag, a vial, a prefilled syringe, or similar container that includes a reconstitution container body defining an inner volume. The inner volume may be sterile. In some approaches, the reconstitution container adapter may also be a CSTD (or, in examples where the prefilled reconstitution container is in the form of a syringe, the container adapter may be a needle) that mates, engages, and/or couples to the vial adapter. Additionally or alternatively, the drug product can be bulk lyophilized and filled into a cartridge or container that is typically used to administer with an IV pump. If needed the dehydrated forms of IVSS, NaCl, and any other components needed for the final administered solution can be bulk lyo'ed and filled into the cassette for long term storage.

The system may be distributed and/or sold as a common kit packaging, but other suitable distribution/packaging is suitable. The drug product may be in the form of a half-life extended bispecific T cell engager (BiTE®), but other drug products are suitable. The diluent include water for injection (“WFI”), but other diluents may be suitable. The containers may be pliable bags, such as IV bags, but other containers may be suitable. In some examples, one or more of the containers is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage.

The system may be used to provide intravenous, subcutaneous, intra-arterial, intramuscular, and/or epidural delivery approaches. By using the system, patient anxiety and or confusion may be reduced due to reduced preparation complexity and wait times caused by the drug preparation process.

In some examples, the prefilled delivery container is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container. Example suitable prefilled delivery containers are described in U.S. Appln. No. 62/804,447, filed on February 12, 2019 and U.S. Appln. No. 62/877,286 filed on Jul. 22, 2019, the contents of each of which are incorporated by reference in their entirety.

One potential challenge associated with administration some therapies is managing an adverse event called “Cytokine Release Syndrome” or CRS. CRS may follow and/or be detected after certain drug therapies. During CRS, a patient's immune reaction may lead to strong and undesirable bodily responses. Currently, CRS is typically or often managed by careful monitoring by a health care professional in a clinical setting. However, remote monitoring may allow patients to manage more of their therapy at home, while potentially maintaining a similar or same level of responsiveness as in a clinical setting. To this end, a wearable cytokine sweat sensor can be used by the patient continuously throughout their therapy to monitor for CRS effects at home. The infusion pump 110 can be connected via a near field Bluetooth signal to this CRS sensor. Should the sensor detected abnormalities that suggest a potential CRS response, it can trigger the pump, alter infusion rate to attempt to mitigate the potential onset of CRS, shutdown automatically and/or prompt an emergency health services call. The closed loop system between the pump and CRS sensor can potentially improve patient safety and overall experience for at home infusion.

FIG. 14 shows an alternative drug delivery assembly 400 (or “system”) for use with the pump 410. For example, the assembly 400 shown in FIG. 14 includes a drug product container 402 which, in the illustrated example, is in the form of an IV bag for containing a drug product 402a (or medicament), an IV input line 404a, an IV output line 404d, a pair of connectors/adaptors 408a, 408b, and the tubing portions 462a, 462d leading to and from the pump 410. As a more specific example, the connection points may include quick-connect sterile connectors with respective sub-components that selectively mate with each other while maintaining sterility or another desirable cleanliness standard. For example, the quick-connect sterile connectors may snap or twist or screw together, they may have sheathed or covered components that become unsheathed or uncovered upon connection, and/or they may have Luer Lock or modified Luer Lock configurations. As another example, the connectors may include one or more stake connectors for coupling one of the tube 462 portions with the drug product container 402. Additionally or alternatively, one or more of the adaptors 408a, 408b may include sensors positioned within or adjacent to respective components of the adapters 408a, 408b. The sensors may be configured to detect pressure changes and/or upper/lower range values that are indicative of a fluid path disconnection event. The assembly 400 also may include a sensor 409 positioned adjacent and/or on the user and configured to measure the user's cytokine levels. The sensor 409 may be in contact with skin of the user and/or may be configured to measure cytokine levels in sweat from the user. In some examples, the sensor 409 may be a wearable sensor. The sensor 409 may be wirelessly connected to the controller 480, and the controller 480 may be configured to stop the drive component (e.g., the motor; not illustrated) upon the sensor 409 measuring a predetermined cytokine level threshold.

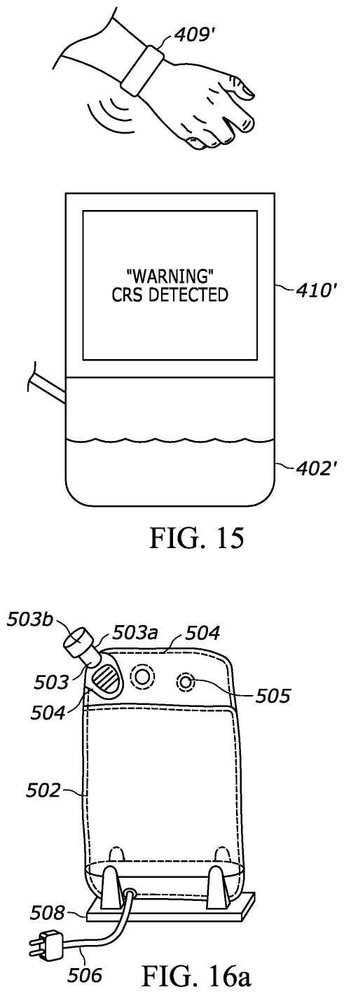

The sensor 409 may be workingly (such as electronically, wirelessly, or via other suitable connection) coupled with the pump controller to stop the drive component and/or alert emergency services or other health care providers upon the sensor measuring a predetermined cytokine level threshold. Additionally or alternatively, should the sensor 409 detect other abnormalities that suggest a potential CRS response, the sensor 409 may trigger the pump 410 to alter infusion rate, shutdown automatically, and/or prompt an emergency health services call. Other examples are possible. FIG. 15 illustrates another such example sensor 409′ in the form of a wearable cytokine sweat sensor that may be worn via a wrist strap or other suitable connection. Alternatively, any suitable sensor and/or configuration may be used.

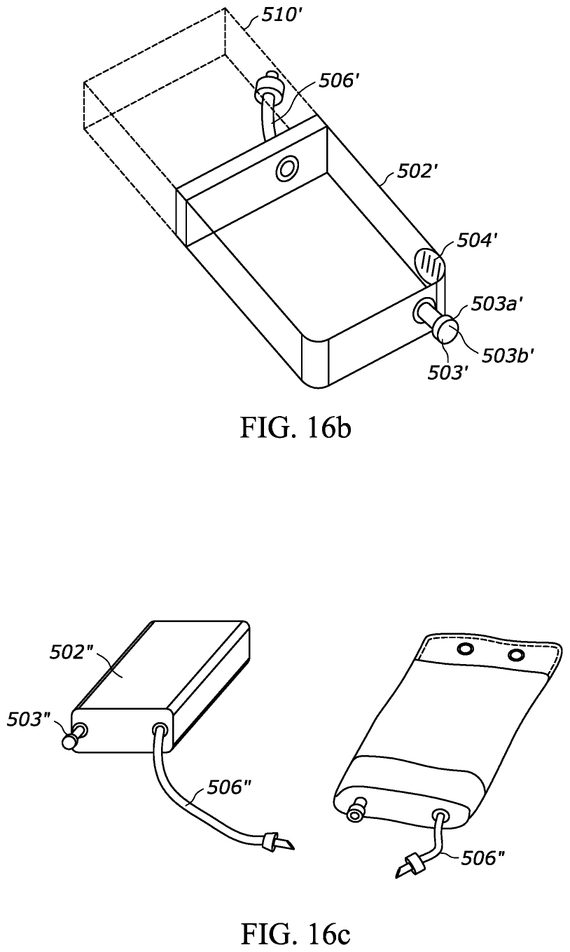

With reference to FIGS. 16a-16c, an alternative drug product container 502 is provided. Some continuous IV-infusion patients may be dissatisfied with IV-spikes and IV-bag ports as they may be overly cumbersome and far-extending (i.e., hard to compress and/or store in small spaces). Further, the spikes and bag ports may be a potential source of leaking for pinch-point occlusions. The alternative drug product container 502 includes a flexible spike-free IV-set attachment port, a sterile-disconnect and pump head integration options, edge or corner mounted Luer-lock, CSTD or FirmClick drug-add port, a sterilized drug add mount/stand for hands free bag manipulation.

As illustrated in FIG. 16a, a drug product container 502 in the form of an IV bag is provided. The drug product container 502 includes a drug-add port 503 having a one-way valve 503a that includes a connector 503b. In some examples, the connector 503b may be in the form of a Luer-lock, a CSTD, or a FirmCLICK compatible syringe coupler. The drug product container 502 further includes a finger grip and corner reinforced area 504 to assist with air removal and handling. In some examples, the contents in the drug product container 502 may flow unimpeded from the one-way valve 503a or when injected into the container 502 via the connector 503b. In other examples, however, the reinforced area 504 may selectively cut off the contents of the container 502 from the drug-add port 503. The drug product container 502 may include reinforced holes 505 for bag-mounting or for securing the drug product container 502 in a hard case (not illustrated). Further, the drug product container 502 may include an IV-set coupler 506 having a sterile-disconnect (or Luer-lock or pump-head integration). This coupler may include one-way valve functionality. The drug product container 502 further includes a stand 508 in which the drug product container 502 may be disposed.

With reference to FIG. 16b, an alternative drug product container 502′ in the form of an IV-cartridge is provided that includes similar features as the drug product container 502. In this example, the drug product container 502′ additionally includes a pump mounting region 510′ having any number of configurations to couple with the drug product container 502′. With reference to FIG. 16c, another alternative drug product container 502″ is provided having any number of similar features as the drug product containers 502′, 502″. In this example, the one-way valve enabled drug-add port 503″ (having a Luer-lock, CSTD, and/or FirmCLICK compatible syringe coupler) may be mounted on the same side as the IV-set coupler 506″.

In some examples, the system may be utilized with medicament in the form of a half-life extended bispecific T cell engager (BiTE®). For example, the active pharmaceutical ingredient (“API”) may be between approximately 2 mcg and approximately 100 mcg, and in some examples, up to approximately 100 mg, depending on the BiTE® and container size, which, may be in a powdered form (i.e., lyophilized) requiring reconstitution. In other examples, the drug product may be in liquid form and may not require reconstitution. Nonetheless, the system includes an accurate quantity of drug product, and thus does not require the need to add additional quantities thereto in a sterile environment. In some examples, the API may be in the form of a half-life extended (“HLE”) BiTE® and/or an IV-admin monoclonal antibody (“mAbs) as desired. These HLE BiTEs® include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BiTE® that is to be administered in a professional healthcare environment.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Raα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery system for delivering a drug product, comprising:
a drug product container containing a drug product;
a fluid path adapted to receive the drug product from the drug product container; and
a drug delivery device positioned along and/or adjacent to the fluid path, the drug delivery device having:
a housing;
a fluid displacement assembly at least partially supported by and/or surrounded by the housing;
a drive component at least partially supported by and/or surrounded by the housing, the drive component adapted to drive the medicament through the fluid displacement assembly; and
a controller workingly coupled with the drive component for controlling the drug delivery device, wherein the controller is adapted to operate in a normal operation mode and a reserve mode.

2. The drug delivery system of claim 1, wherein in the reserve mode, the controller causes the drug product to be dispensed at a ramp-down rate.

3. The drug delivery system of claim 1, wherein the controller is adapted to operate in the normal operation mode for a predetermined time.

4. The drug delivery system of claim 1, wherein the drug product container contains a reserve quantity of drug product to operation in the reserve mode.

5. The drug delivery system of claim 1, wherein the controller further includes at least one of an alarm or a feedback mechanism to alert a user upon initiation of the reserve mode.

6. A drug delivery system for delivering a drug product, comprising:
a drug product container containing a drug product;
a fluid path adapted to receive the drug product from the drug product container; and
a drug delivery device positioned along and/or adjacent to the fluid path, the drug delivery device having:
a housing;
a fluid displacement assembly at least partially supported by and/or surrounded by the housing;
a drive component at least partially supported by and/or surrounded by the housing, wherein the drive component is adapted to drive the medicament through the fluid displacement assembly; and
a controller workingly coupled with the drive component for controlling the drug delivery device, wherein the controller is adapted to send a user output when a low drug event has occurred.

7. A drug delivery system for delivering a drug product, comprising:
a drug product container containing a drug product;
a fluid path adapted to receive the drug product from the drug product container;
a drug delivery device positioned along and/or adjacent to the fluid path; and
a scannable identifier tag coupled with at least the drug product container, the fluid path, and/or the drug delivery device, the scannable identifier tag adapted to provide information regarding the drug product container, the fluid path, and/or the drug delivery device.

8. The drug delivery system of claim 7, wherein the scannable identifier tag coupled with the drug product container is adapted to provide information selected from the following list: drug product date of manufacture, drug product expiration date, drug product volume, drug product lot number, drug product model number, drug product region, and/or drug product temperature information.

9. The drug delivery system of claim 7, wherein the scannable identifier tag coupled with the drug delivery device is adapted to provide information selected from the following list: authenticating information, whether a cartridge is new, volume of drug product delivered, expiration information, desired priming volumes, desired flow rates, therapy log information, whether IV lines are new, and/or whether IV lines are compatible.

10. The drug delivery system of claim 7, wherein the scannable identifier tag coupled with the fluid path is adapted to provide information selected from the following list: IV line date of manufacture, IV line expiration date, IV line lot number, IV line model number, IV line region, IV line length and/or IV line time in use.

11. A device for storing a drug product container in a temperature-controlled state, comprising:
a container positionable in at least an open configuration and a closed configuration;
a temperature element adjustable between a heating mode and a cooling mode to selectively heat and/or cool the container; and
an ultraviolet element adapted to reduce a bioburden level of at least a portion of the container.

12. A drug delivery system for delivering a medicament to a user, comprising:
a drug product container containing a medicament;
a fluid path adapted to at least selectively fluidly connected the drug product container and the user;
a sensor positioned adjacent and/or on the user and adapted to measure cytokine levels; and
a drug delivery device positioned adjacent to and/or along the fluid path, the drug delivery device having:
a housing;
a pump coupled with the housing;
a drive component for driving the medicament through the pump; and
a controller workingly coupled with the drive component, wherein the controller is adapted to adjust at least one parameter of the drive component based on input information received from the sensor.

13. The drug delivery system of claim 12, wherein at least a portion of the controller is in contact with skin of the user.

14. The drug delivery system of claim 12, wherein the sensor is adapted to measure a cytokine level in sweat from the user.

15. The drug delivery system of claim 12, wherein the sensor comprises a wearable sensor.

16. The drug delivery system as in claim 12, wherein the sensor is wirelessly connected to the controller.

17. The drug delivery system as in claim 12, wherein the controller is adapted to stop the drive component upon the sensor measuring a predetermined cytokine level threshold.

18. A flexible drug product container for a drug delivery system having an intravenous tube, the drug product container comprising:
a body portion adapted to contain a drug product;
a spike-free IV-set attachment port; and
a sterile-disconnect and custom pump head integration component.
